(19) United States
(12) Patent Application Publication
(10) Pub. No.: US 2012/0035687 A1
(43) Pub. Date: Feb. 9, 2012

(54) IMPLANTABLE ELECTRICAL STIMULATOR

(75) Inventors: Shey-Shi LU, TAIPEI (TW); Hung-Wei CHIU, Taipei City (TW); I-Hsiu HO, Taipei City (TW); Po-Hsiang FANG, TAIPEI (TW); Chang-Lun WANG, TAIPEI (TW); Yi-Chin LEE, TAIPEI (TW); Chii-Wann LIN, TAIPEI (TW); Mu-Lien LIN, Taipei City (TW); Chi-Heng Chang, Taoyuan County (TW); Yao-Chuan Tsai, TAIPEI (TW); Yeong-Ray Wen, Taipei City (TW); Wen-Pin Shih, Taipei City (TW); Yao-Joe Yang, TAIPEI (TW); Wei-Tso LIN, TAIPEI (TW)

(73) Assignee: NATIONAL TAIWAN UNIVERSITY, TAIPEI (TW)

(21) Appl. No.: 13/015,589

(22) Filed: Jan. 28, 2011

(30) Foreign Application Priority Data
Aug. 6, 2010 (TW) 099126334

(51) Int. Cl. A61N 1/378

(52) U.S. Cl. 607/61

(57) ABSTRACT

Disclosed herein is an implantable electrical stimulator which includes two stimulating electrodes, a system-on-chip and an inductive coil. The system-on-chip can apply electric stimulation to the dorsal root ganglion via the stimulating electrodes. An external power supply can wirelessly charge the system-on-chip through the inductive coil.

### RELATED APPLICATIONS

This application claims priority to Taiwan Application Serial Number 099126334, filed Aug. 6, 2010, which is herein incorporated by reference.

### BACKGROUND

1. Field of Invention

The present invention relates to an electronic device. More particularly, the present invention relates to an electrical stimulator.

2. Description of Related Art

Nerve dysfunctions belong to a main category of neurological diseases. Although pain is interpreted as the fifth vital sign by many professions, the presence of different degrees of pain significantly affects quality of life for many patients, especially the elderly.

Current treatments to these neurological diseases are quite complicated. For example, acupuncture electrodes connecting to bulky medical apparatus are inserted in to the body of the subject during each treatment. The insertion of the acupuncture electrodes may cause pain to the subject, and increase the possibility of infections.

In view of the foregoing, there is an urgent need in the related field to provide a way to reduce pain to the subject.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present invention is directed to implantable electric stimulator adapted to be implanted into the body a subject.

According to one embodiment of the present invention, the implantable electric stimulator comprises two stimulating electrodes, a system-on-chip and an inductive coil. In structure, the system-on-chip electrically connects the stimulating electrodes, and the inductive coil is electrically connected to the system-on-chip. In operation, an external power supply may wirelessly charge the system-on-chip via the inductive coil, whereas the system-on-chip may apply electric stimulation to a dorsal root ganglion through the stimulating electrodes. As such, it is possible to ameliorate the pain by applying electric stimulation to the dorsal root ganglion.

According to another embodiment of the present invention, the implantable electric stimulator comprises two stimulating electrodes, a system-on-chip, and a receiving coil. In operation, the stimulating electrodes is electrically connected to a dorsal root ganglion, the receiving coil is inductively coupled to the output coil of an external power supply so that the external power supply is operable to wireless charge the system-on-chip, and the system-on-chip outputs electric stimulation via the stimulating electrodes. As such, the pain of the subject being treated could be alleviated when the dorsal root ganglion are subjected to electric stimulation.

In view of the foregoing, the technical solution provided by the present disclosure exhibits obvious advantages and beneficial effects as compared with conventional techniques. The technical solution embodies substantial technical progress and provides a wide range of industrial utilities. The advantages provided by the present disclosure include:

1. The system-on-chip is wireless charged, and hence, no plug sockets or batteries are required for charging; as such, the present implantable electrical stimulator is portable and easy-to-use; and

2. The functionality of the stimulator is embodied in the system-on-chip thereby miniaturizing the volume of the present implantable electric stimulator so that it is suitable to be implanted in to human body; as such, patients would no longer suffer from the uncomfortable cause by the insertion of the acupuncture electrodes during each treatment.

Many of the attendant features will be more readily appreciated as the same becomes better understood by reference to the following detailed description considered in connection with the accompanying drawings.

### BRIEF DESCRIPTION OF THE DRAWINGS

The present description will be better understood from the following detailed description read in light of the accompanying drawings, wherein:

FIG. 1 is a schematic diagram illustrating an implantable electric stimulator according to one embodiment of the present disclosure;

FIG. 2 is a circuit diagram of the rectifier depicted in FIG. 1 according to one embodiment of the present disclosure;

FIG. 3 is a circuit diagram of the voltage limiter depicted in FIG. 1 according to one embodiment of the present disclosure;

FIG. 4 is a circuit diagram of the regulator depicted in FIG. 1 according to one embodiment of the present disclosure;

FIG. 5 is a circuit diagram of the clock regenerator depicted in FIG. 1 according to one embodiment of the present disclosure;

FIG. 6 is a circuit diagram of the radio frequency receiver depicted in FIG. 1 according to one embodiment of the present disclosure;

FIG. 7 is a circuit diagram of the power-on reset circuit depicted in FIG. 1 according to one embodiment of the present disclosure;

FIG. 8 is a circuit diagram of the driver depicted in FIG. 1 according to one embodiment of the present disclosure; and

FIG. 9 is a time sequence diagram of the pulse signal outputted by the driver depicted in FIG. 1 according to one embodiment of the present disclosure.

Like reference numerals are used to designate like parts in the accompanying drawings.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples. Also, well-known elements and/or steps are not discussed in the embodiments in detail for the sake of clarity and brevity.

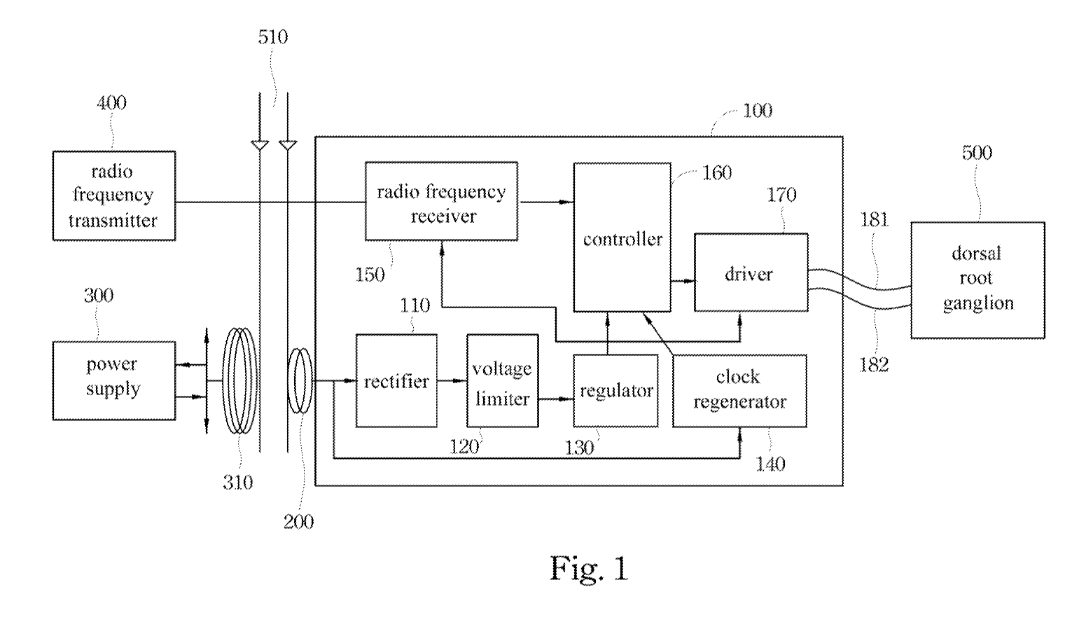

FIG. 1 is a schematic diagram illustrating an implantable electric stimulator according to one embodiment of the present disclosure. As shown in FIG. 1, the implantable electric stimulator may include a system-on-chip 100, an inductive coil (receiving coil) 200, and two stimulating electrodes 181, 182. The implantable electric stimulator may be implanted into the human body and is positioned under the skin 510 so as to stimulate the dorsal root ganglion 500 of the spine.

In structure, the inductive coil 200 is electrically connected to the system-on-chip 100, the system-on-chip 100 electrically connects the stimulating electrodes 181, 182, and the stimulating electrodes 181, 182 are used for electrically connecting the dorsal root ganglion 500. In operation, the external power supply 300 may wirelessly charge the system-on-chip 100 via the inductive coil 200, and the system-on-chip 100 may apply electrical stimulation to the dorsal root ganglion 500 through the stimulating electrodes 181, 182. Dorsal root ganglion 500 is a nodule on a dorsal root that contains cell bodies of neurons of peripheral nervous system which is responsible for transmitting sensory information along each of the peripheral axons. The sensory information is electrochemical signals representing senses of touch, pain, temperature, etc. The sensory information is then integrated by the central nervous system so that the brain may perceive the specific sense. As such, it is possible to alleviate the pain by electrically stimulating the dorsal root ganglion 500.

In practice, the power is transferred by means of the mutual induction between the inductive coil (output coil) 310 of the external power supply 300 and the inductive coil (receiving coil) 200 of the implantable electric stimulator. In the field of the wireless power transmission, one of the most important concerns is to improve the efficiency of the power transmission. As such, in one embodiment, the external power supply 300 may include a Class-E power amplifier, which may provide higher power transmission efficiency as compared with other types of power amplifiers. Accordingly, problems such as the wireless signal is too weak to be recognized or the power is not sufficient for the system-on-chip 100 can be avoided.

Also, the choice of the frequency band at least depends on the distance that the wireless signal should traverse in the human tissue. Generally, the high-frequency signals have shorter depth of penetration in human body, as compared with short-frequency signals. As such, in one embodiment, the frequency of the wireless signal is about 1 MHz.

As shown in FIG. 1, the system-on-chip 100 may include elements such as: a rectifier 110, a voltage limiter 120, a regulator 130, a clock regenerator 140, a radio frequency receiver 150, controller 160 and driver 170. In the present embodiment, the above-identified elements are integrated into the system-on-chip 100 so as to miniaturize the volume of the implantable electric stimulator.

It should be appreciated that by manufacturing the above-identified elements as individual chips and disposing such chips on a circuit board, the chips should be packaged separately. As such, the chips (elements) may occupy extra space (as compared with the integrated elements provided in the present embodiment), and additional wirings are required to connect these chips, thereby increasing the volume of the electric stimulator. However, larger electric stimulators may raise the chances of infections of the subjects and cause uncomfortableness to the subjects.

In addition, the system-on-chip 100 may be encapsulated by a bio-compatible material so as to facilitate the implantation. For example, the bio-compatible material may be Poly (dimethylsiloxane) (PDMS). PDMS can be used as a protection layer to seal the system-on-chip 100. Analysis performed by the present inventor shows that this encapsulation exhibits satisfactory hermeticity, tensile property, and flexibility. Also, this encapsulation can readily adhere to the human tissue and provide suitable strength.

The mechanism of the electric energy conversion of the employed by the system-on-chip 100 is implanted by the rectifier 110, the voltage limiter 120, and the regulator 130. In structure, the controller 160 is electrically connected to the regulator 130, the regulator 130 is electrically connected to the voltage limiter 120, the voltage limiter 120 is electrically connected to the rectifier 110, and the rectifier 110 is electrically connected to the inductive coil 200.

In operation, the wireless signal provided from the external power supply 300 may be rectified by the rectifier 110 to obtain a direct current through the induction coupling between the inductive coil 200 and the inductive coil 310 of the external power supply 300. Then, the voltage limiter 120 may limit the voltage of the direct current to a value lower than a predetermined voltage so that the voltage would not exceed the system load. Afterwards, the regulator 130 may regulate the direct current to obtain a steady voltage, remove the noise, and provide the steady voltage to the controller 160 so that the controller 160 has sufficient electric energy to generate a stimulus signal. In the present embodiment, in order to improve the driving force outputted by the controller 160, and avoid the distortions of the stimulus signal, a driver 170 is employed to enhance the stimulus signal, and the enhanced stimulus signal is outputted to the dorsal root ganglion 500 via the stimulating electrodes 181, 182.

Specifically, the mechanism of providing the electric stimulation may be implanted by the clock regenerator 140 in conjunction with the controller 160 and the driver 170. In structure, the stimulating electrodes 181, 182 are electrically connected to the driver 170, the driver 170 is electrically connected to the controller 160, the controller 160 is electrically connected to the clock regenerator 140, and the clock regenerator 140 is electrically connected to the is inductive coil 310.

In operation, the wireless signal provided from the external power supply 300 is converted into working clock(s) for the controller 170 through the induction coupling between the inductive coil 200 and the inductive coil 310 of the external power supply 300, and thereby, the controller 160 may generate stimulus signal(s) based on the working clock(s). Thereafter, the stimulus signal, after being enhanced by the driver 170, is outputted to the dorsal root ganglion 500 via the stimulating electrodes 181, 182.

In addition, a modulated parameter instruction may be provided to the system-on-chip 100 through an external radio frequency transmitter 400, so as to control the waveform outputted by the implantable electric stimulator. In one embodiment, the above-mentioned mechanism may be implanted by the collaboration of the radio frequency receiver 150 and the controller 160. In structure, the controller 160 is electrically connected to the receiver 150, and the receiver 150 may wirelessly communicate with the radio frequency transmitter 400.

In operation, when the modulated signal transmitted by the radio frequency transmitter 400 penetrates the skin 510 and reaches the system-on-chip 100, the radio frequency receiver 150 may obtain and demodulate the modulated signal to output a demodulated signal so that the controller 160 may set the parameter(s) for the stimulus signal based on the demodulated signal. For example, if the stimulus signal is a pulse, the parameter thereof may be the carrier frequency, the cycle time (period) and/or the duty cycle, etc.; whereas if the stimulus signal is a sine wave, the parameter thereof may be the cycle (period) and/or the amplitude, etc.

In one embodiment, the radio frequency transmitter 400 and the external power supply 200 may be integrated in a single electronic device, such as a cellular phone or other portable electronic devices. As such, the wireless charging of the implantable electric stimulator and the intensity and duration of the electric stimulation may be achieved simply by operating the cellular phone.

In practice, the circuit framework of the system-on-chip 100 is embodied by the 0.35 micrometer CMOS process by TSMC. In such circuit framework, the efficiency of the conversion from the wireless signal into the direct current is about 80%, the wave amplitude of the radio frequency transmission may be no less than 3 V, the frequency of the wireless signal provided by the external power supply 300 is about 1 MHz, the frequency of the modulated obtained by the signal radio frequency receiver 150 is about 402 MHz, the sensitivity of the radio frequency receiver 150 is about −62 dBm, the voltages outputted by the stimulating electrodes 181, 182 are limited to 5 V at maximum, whereas the voltage of about 3 V is sufficient to substantially alleviate or relive the pain.

Also, since the proteins of the human body may start to denature at about 41′C, the operating temperature of the system-on-chip 100 should not exceed 39′C. The size of the system-on-chip 100 is about 2.159 mm*2.146 mm which is suitable for being implanted into the human body.

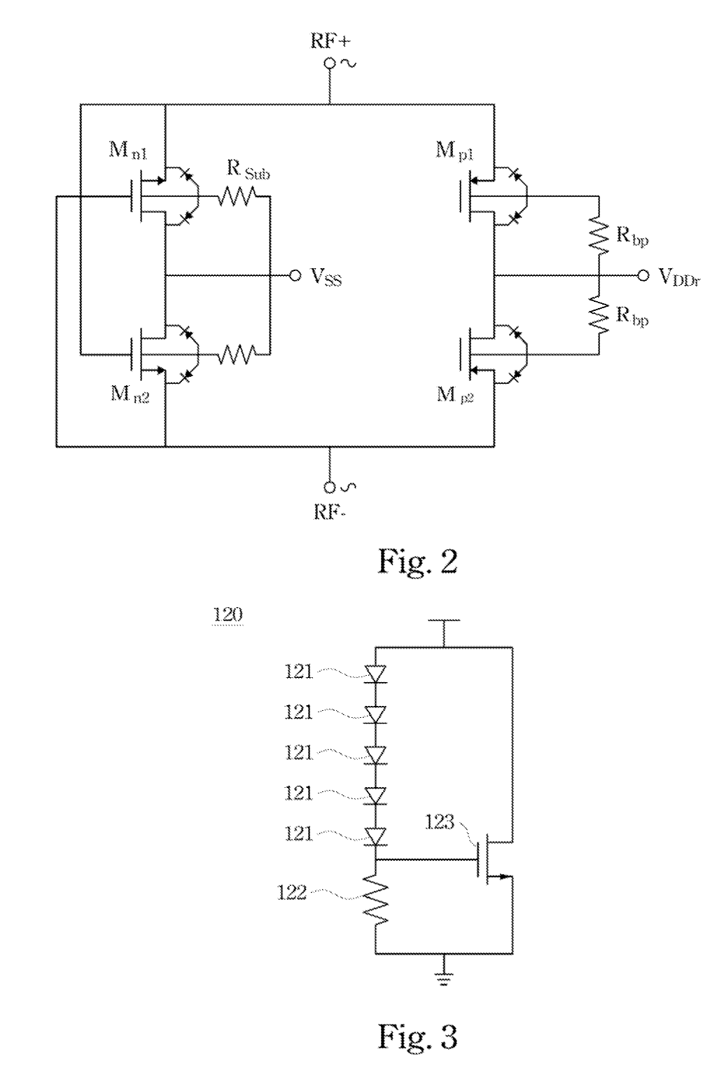

Detailed descriptions of each of the elements illustrated in in system-on-chip 100 are provided hereinbelow in connection with FIG. 2 to FIG. 8 so as to facilitate the understanding to the above-mentioned circuit framework.

FIG. 2 is a circuit diagram of the rectifier 110 depicted in FIG. 1 according to one embodiment of the present disclosure. As shown in FIG. 2, the rectifier 110 includes transistors, P-type metal oxide semiconductors Mp1, Mp2 and N-type metal oxide semiconductors Mn1, Mn2, which are disposed and connected as a diode.

In the present embodiment, the P-type metal oxide semiconductors and the N-type metal oxide semiconductors are connected as a backward diode and assembled as a bridge-type full wave rectification. When the wireless signals are inputted from the two terminals of the differential motion, a full wave voltage may be generated at the output, wherein the rectification is achieved mostly by the diode formed at the P-N junction between the source and the body structure. The advantage of such framework lies in that only metal oxide semiconductors are required to implant the functionality of the rectification.

FIG. 3 is a circuit diagram of the voltage limiter 120 depicted in FIG. 1 according to one embodiment of the present disclosure. As shown in FIG. 3, the voltage limiter 120 includes a plurality of diodes 121, a resistor 122 and a P-type metal oxide semiconductor 123.

In operation, a voltage limiter 120 is disposed behind the rectifier 110 to prevent the damage to the circuit caused by the instantaneous conducted current or voltage that are higher than a predetermined level. When the output voltage exceeds the predetermined level, the diode(s) 121 of the voltage limiter 120 would be conducted and limit the output voltage under a predetermined voltage. The value of the predetermined voltage depends on the number of the diodes 121 serial-connected. Moreover, each of the diodes 121 is implemented by the P-type metal oxide semiconductor in the form of a diode.

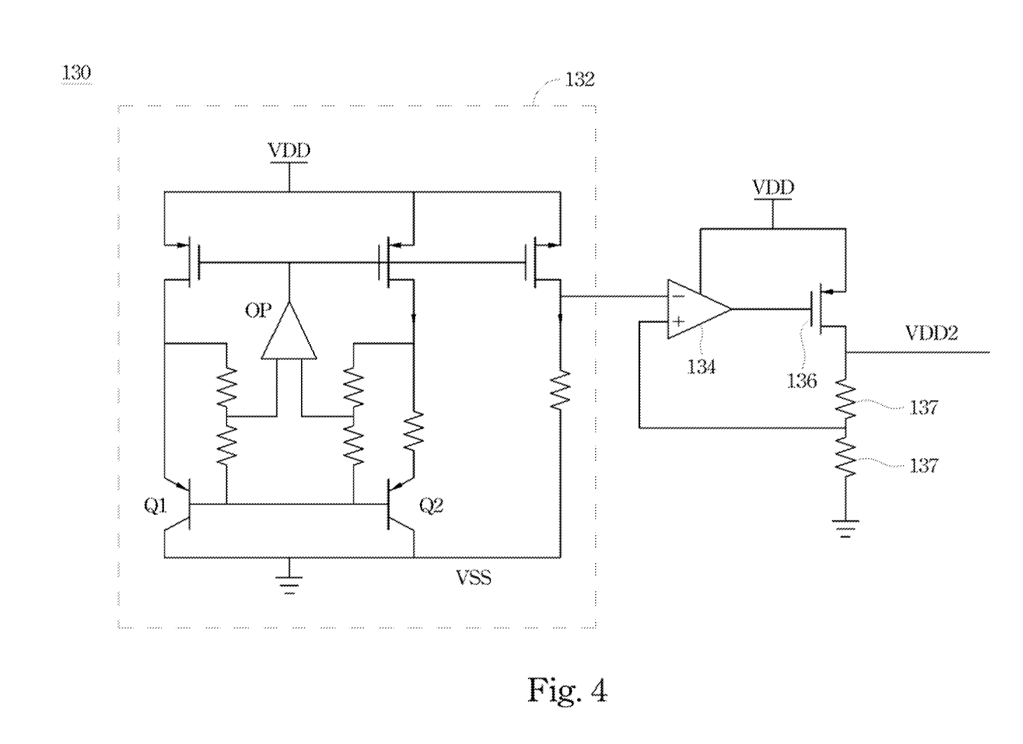

FIG. 4 is a circuit diagram of the regulator 130 depicted in FIG. 1 according to one embodiment of the present disclosure. In the present embodiment, the regulator 130 is a low-dropout regulator. In practice, small volume and low power consumption are requisites to the present system-on-chip of the implantable electric stimulator. Hence, as compared with the general switching regulators and direct-current-to-direct-current converters, the low-dropout regulator used herein is advantageous in that the response time of the outputted voltage to the variation of the inputted voltage or load is faster, the ripple and noise of the outputted voltage is lower, and the circuit architecture is simpler. Also, the size of the present electric stimulator could be miniaturized, and the manufacturing cost could be reduced. Also, it should be noted that the intrinsic properties of the present low-dropout regulator (such as the quiescent current, voltage drop and noise) is significantly enhanced by the present design where the low-dropout regulator is manufactured by a CMOS process that provide a compact product with low manufacturing cost.

As shown in FIG. 4, the low-dropout regulator 130 includes an energy gap reference voltage circuit 132 and a voltage regulator. In structure, the energy gap reference voltage circuit 132 is electrically connected to the voltage regulator. In operation, the voltage regulator received the voltage outputted from the voltage limiter 120 and regulates the desired steady direct voltage (such as, 3 V) for use as the energy source for the rest segments of the chip.

In practice, the voltage regulator includes a lock loop consisting of an amplifier 134 in conjunction with metal oxide semiconductor field effect transistor 135 and resistors 136, 137. The voltage regulator 133 requires an accurate reference voltage, and as such, an energy gap reference voltage circuit 132 is employed in the present embodiment to generate a steady power source that would not shift with the temperature variation.

Conventionally, the elevation or drop of the temperature may affect the parameters for the semiconductor manufacturing process so that the initially designed voltage and current would shift. However, the energy gap reference voltage circuit 132 is designed to get rid of the effects caused by temperature by using semiconductor elements having different positive and negative temperature coefficients to mutually offset the temperature effects. To conventional CMOS processes, resistors and metal oxide semiconductor field effect transistor have positive temperature coefficients; that is, the resistance value of the resistor and the threshold voltage of the semiconductor field effect transistor would increase as the temperature increase, which is disadvantageous to CMOS processes. As such, a direct solution to this disadvantage is to use the material(s) currently used in the process to form diodes or bipolar transistors Q1, Q2, so that the material(s) having a negative temperature coefficient is operable to compensate the temperature variation.

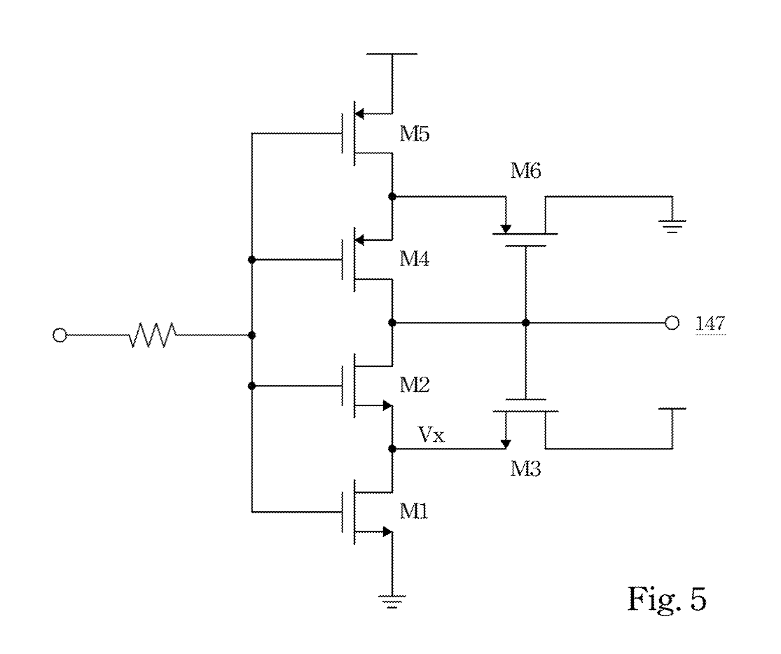

FIG. 5 is a circuit diagram of the clock regenerator 140 depicted in FIG. 1 according to one embodiment of the present disclosure. As shown in FIG. 5, the clock regenerator 140 consists essentially of metal oxide semiconductor field effect transistors M1, M2, M3, M4, M5, and M6. In operation, the clock regenerator 140 may convert wireless signal having sine waveforms into a working clock having a square waveform, and then output the working clock to the controller through the output terminal 147.

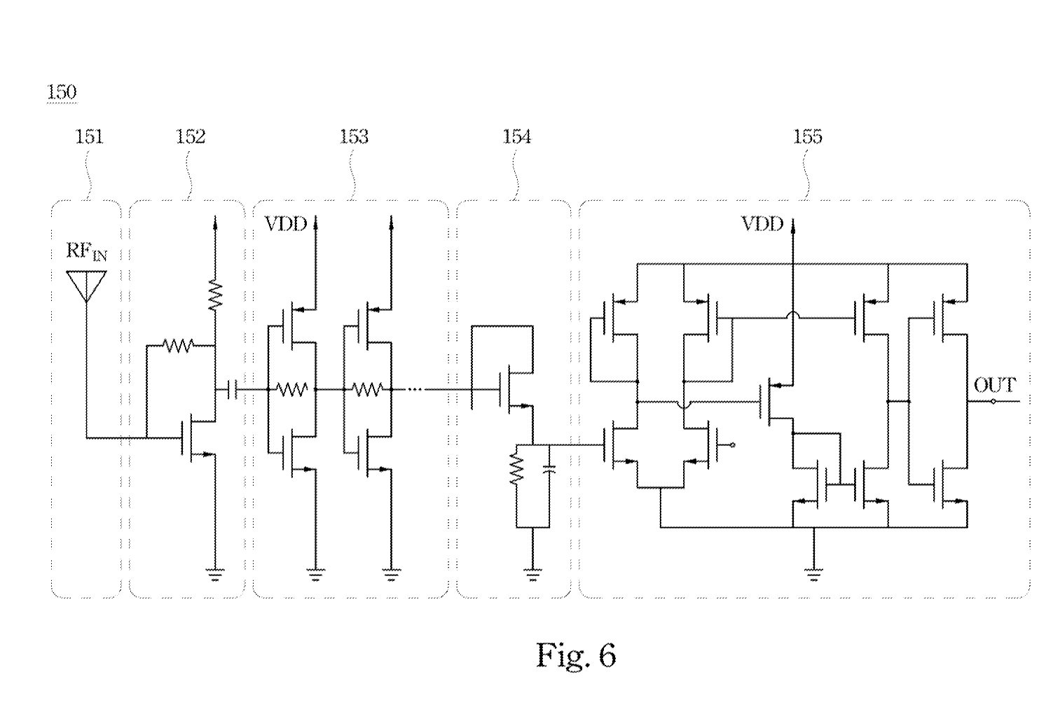

FIG. 6 is a circuit diagram of the radio frequency receiver 150 depicted in FIG. 1 according to one embodiment of the present disclosure. As shown in FIG. 6, the radio frequency receiver 150 includes a radio frequency antenna 151, a head amplifier 152, a cascade amplifier 153, an envelope detector 154, and a comparator/buffer circuit 155.

In structure, the radio frequency antenna 151 is electrically connected to the head amplifier 152, the head amplifier 152 is electrically connected to the cascade amplifier 153, the cascade amplifier 153 is electrically connected to the envelope detector 154, and the envelope detector 154 is electrically connected to the comparator/buffer circuit 155.

In operation, the radio frequency antenna 151 may receive a modulated signal from the radio frequency transmitter 400, the amplifiers 152 and 153 may amplify the modulated signal, the envelope detector 154 may detect the envelope of the amplified modulated signal to output a detected signal for the comparator disposed at the front end of the circuit 155 to determine the voltage level of the detected signal thereby obtaining a demodulated signal, and the buffer disposed at the rear end of the circuit 155 outputs the demodulated signal to the controller 160 shown in FIG. 1.

The envelope detector 154 characterized in that the current and voltage of its circuit are relatively steady when the power source is shifted significantly, and hence the current and voltage would not greatly vary depending on the shift of the power source. As such, it is possible to generate a detected signal having a relatively steady direct current level by using the modulated signal as an inputting power source voltage, thereby accomplishing the functionality for detecting the envelope of the modulated signal.

The voltages of the detected signal generated by the envelope detector 154 would have overlapping portions, and hence, a comparator is used to determine the voltage level of the detected signal thereby obtaining a demodulated signal. The buffer is disposed at the rear end of the circuit 155. The controller 160 is connected to the back end of the output terminal (OUT), and hence, the driving force of the output should be increase to avoid the distortion of the signal.

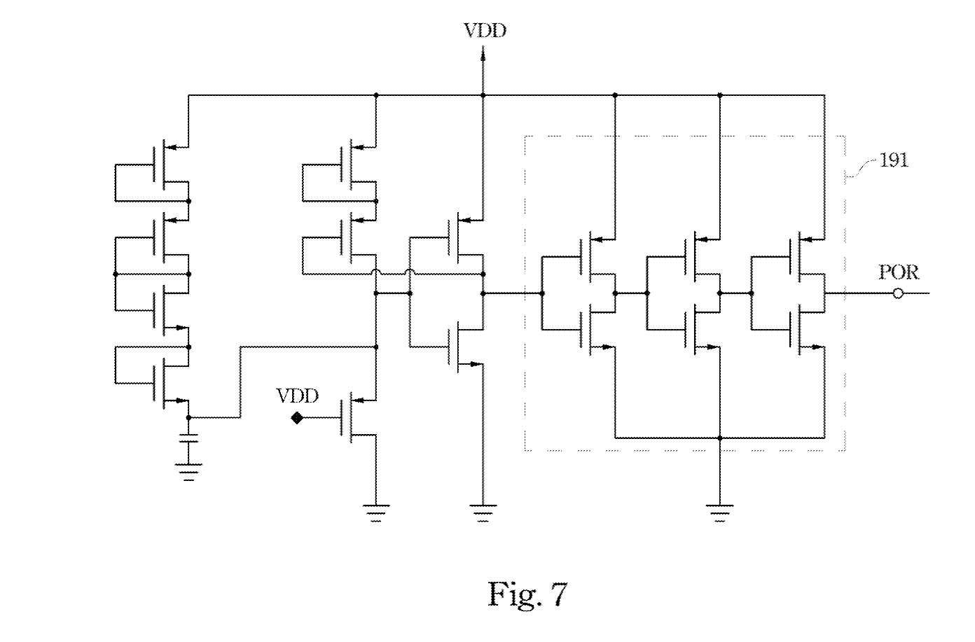

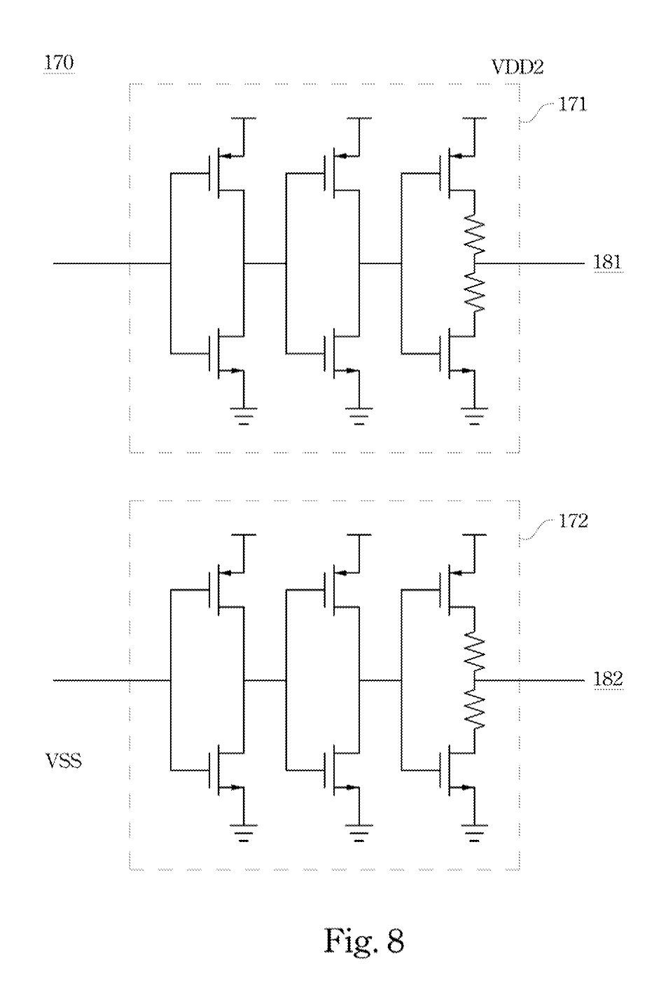

FIG. 7 is a circuit diagram of the power-on reset circuit 190 depicted in FIG. 1 according to one embodiment of the present disclosure. In structure, the power-on reset circuit 190 may be integrated into the system-on-chip 100, and electrically connected to the controller 160 shown in FIG. 1. In operation, when the wireless charging is carried out by the external power supply 300, the power-on reset circuit 190 may reset the controller 160. In the present embodiment, the power-on reset circuit 190 has a set of inverters 191 for increasing the driving force of the output, and a resetting signal is outputted to the controller shown in FIG. 1.

FIG. 8 is a circuit diagram of the driver 170 depicted in FIG. 1 according to one embodiment of the present disclosure. As shown in FIG. 8, the driver 170 includes a first set of inverters 171 and a second set of inverters 172. In structure, the first set of inverters 171 is electrically connected to the stimulating electrode 181, whereas the second set of inverter 172 is electrically connected to the stimulating electrode 182. In operation, since the stimulating electrodes 181, 182 are connected to the dorsal root ganglion 500, it is required to increase the driving force of the output by the driving circuits consisting of inverters, so as to avoid the distortion of the stimulus signal being transferred into the human body.

Moreover, the controller 160 shown in FIG. 1 may be a logic controller, digital controller, logic control circuit, programmable logic controller, programmable digital controller or the same. The controller 160 may have a pulse-width modulating device. The pulse-width modulating device may periodically output at least one pulse for use as the stimulus signal, and then the pulse signal is outputted by the driver 170.

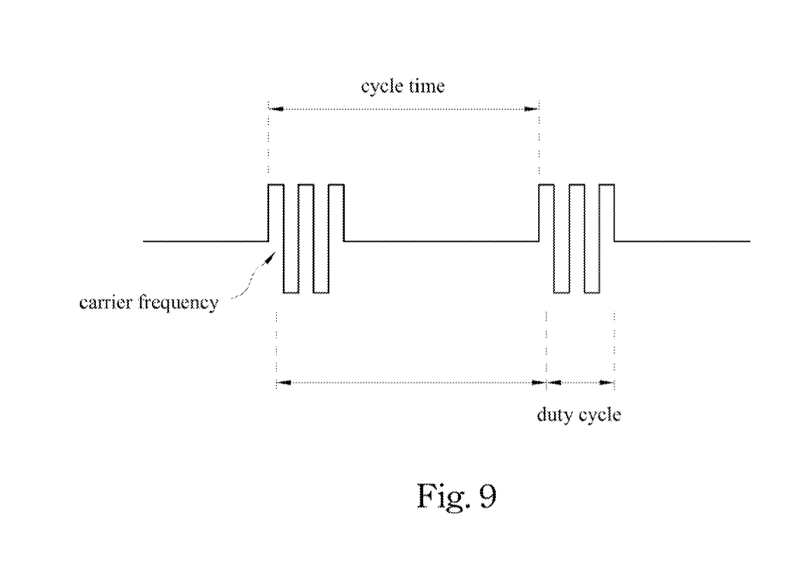

FIG. 9 is a time sequence diagram of the pulse signal outputted by the driver 170 depicted in FIG. 1 according to one embodiment of the present disclosure. In practice, the carrier frequency of the pulse signal is in the range of about 4 kHz to about 1 MHz, and the cycle time thereof is about 0.05 seconds to about 1.25 seconds, and the duty cycle may be adjusted in the range from 0% to 100%. In other embodiments, the waveform of the stimulus signal generated by the controller 160 may be a sine wave, triangular wave or other mixed wave.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An implantable electric stimulator, comprising:
two stimulating electrodes;
a system-on-chip electrically connecting the stimulating electrodes, for applying electric stimulation to a dorsal root ganglion via the stimulating electrodes; and
an inductive coil electrically connected to the system-on-chip such that an external power supply is operable to wirelessly charge the system-on-chip through the inductive coil.

2. The implantable electric stimulator of claim 1, wherein the system-on-chip comprises:
a controller for generating a stimulus signal; and
a driver electrically connected to the stimulating electrodes, for enhancing the stimulus signal such that the stimulating electrodes is operable to output the enhanced stimulus signal to the dorsal root ganglion.

3. The implantable electric stimulator of claim 2, wherein the system-on-chip further comprises:
a clock regenerator electrically connected to the inductive coil, for converting a wireless signal provided by the external power supply into a working clock for the controller such that the controller is operable to generate the stimulus signal based on the working clock.

4. The implantable electric stimulator of claim 2, wherein the system-on-chip further comprising:
a radio frequency receiver electrically connected to the controller, for receiving and demodulating a modulated signal from a radio frequency transmitter to output a demodulated signal such that the controller is operable to set the parameter of the stimulus signal based on the demodulated signal.

5. The implantable electric stimulator of claim 4, wherein the radio frequency receiver comprises:
at least one amplifier for amplifying the modulated signal;
an envelope detector for detecting the envelope line of the amplified modulated signal to output a detected signal;
a comparator for discriminating the potential of the detected signal to obtain the demodulated signal; and
a buffer for outputting the demodulated signal to the controller.

6. The implantable electric stimulator of claim 2, wherein the system-on-chip further comprising:
a rectifier electrically connected to the inductive coil, for rectifying the wireless signal provided by the external power supply into a direct current;
a voltage limiter electrically connected to the rectifier, for limiting the voltage of the direct current to a value lower than a predetermined voltage; and
a regulator electrically connected to the voltage limiter, for regulating the direct current to provide a steady voltage, and providing the steady voltage to the controller.

7. The implantable electric stimulator of claim 6, wherein the regulator is a low-dropout regulator.

8. The implantable electric stimulator of claim 2, wherein the system-on-chip further comprises:
a power-on reset circuit for resetting the controller when the external power supply is wirelessly charging the system-on-chip.

9. The implantable electric stimulator of claim 2, wherein the controller comprises:
a pulse width modulating device for periodically outputting at least one pulse as the stimulus signal.

10. The implantable electric stimulator of claim 1, further comprising:
a bio-compatible material encapsulating the system-on-chip.

11. An implantable electric stimulator, comprising:
two stimulating electrodes adapted to electrically connect a dorsal root ganglion;
a system-on-chip for outputting electric stimulation via the stimulating electrodes; and
a receiving coil for inductively coupled to the output coil of an external power supply so that the external power supply is operable to wireless charge the system-on-chip.

12. The implantable electric stimulator of claim 11, wherein the system-on-chip comprises:
a controller;
a clock regenerator electrically connected to the receiving coil, for converting the wireless signal provided by the external power supply into a working clock for the controller;
a rectifier electrically connected to the receiving coil, for rectifying the wireless signal provided by the external power supply into a direct current;
a voltage limiter electrically connected to the rectifier, for limiting the voltage of the direct current to a value lower than a predetermined voltage;
a regulator electrically connected to the voltage limiter, for regulating the direct current to provide a steady voltage, and providing the steady voltage to the controller so that the controller generates a stimulus signal based on the working clock; and
a driver electrically connected to the stimulating electrodes, for enhancing the stimulus signal so that the stimulating electrodes output the enhanced stimulus signal to the dorsal root ganglion.

13. The implantable electric stimulator of claim 12, wherein the system-on-chip further comprising:
a power-on reset circuit for resetting the controller when the external power supply is wirelessly charging the system-on-chip.

14. The implantable electric stimulator of claim 12, wherein the regulator is a low-dropout regulator.

15. The implantable electric stimulator of claim 12, wherein the system-on-chip further comprising:
a radio frequency receiver electrically connected to the controller, for receiving and demodulating a modulated signal from a radio frequency transmitter to output a demodulated signal such that the controller is operable to set the parameter of the stimulus signal based on the demodulated signal.

16. The implantable electric stimulator of claim 15, wherein the radio frequency receiver comprises:
at least one amplifier for amplifying the modulated signal;
an envelope detector for detecting the envelope line of the amplified modulated signal to output a detected signal;
a comparator for discriminating the potential of the detected signal to obtain the demodulated signal; and
a buffer for outputting the demodulated signal to the controller.

17. The implantable electric stimulator of claim 12, wherein the controller comprises:
a pulse width modulating device for periodically outputting at least one pulse as the stimulus signal.

18. The implantable electric stimulator of claim 11, further comprising:
a bio-compatible material encapsulating the system-on-chip.
